(19) United States
(12) Patent Application Publication
(10) Pub. No.: US 2022/0402861 A1
(43) Pub. Date: Dec. 22, 2022

(54) A Crystalline form of (R)-terbutaline Hydrochloride

(71) Applicant: WEN TAN, Foshan City (CN)

(72) Inventor: WEN TAN, Foshan City (CN)

(21) Appl. No.: 17/607,893

(22) PCT Filed: Mar. 1, 2021
(86) PCT No.: PCT/US21/20175

§ 371 Date: Oct. 31, 2021

Related U.S. Application Data
(60) Provisional application No. 62/983,772, filed on Mar. 2, 2020.

(51) Int. Cl. C07C 215/60

(52) U.S. Cl. CPC C07C 215/60; C07B 2200/13

(57) ABSTRACT

The present invention relates to a novel crystalline form of (R)-terbutaline hydrochloride salt, its process and its therapeutically uses.

### CROSS-REFERENCE TO RELATED APPLICATIONS

The present application claims priority to U.S. Provisional Patent Application No. 62/844,398, filed May 7, 2019, which are herein incorporated by reference in their entirety.

This application is also a continuation of International Application No. PCT/US20/31539, filed May 5, 2020.

This application is also a continuation-in-part of International Application No. PCT/US19/50120. Filed Sep. 7, 2019.

### FIELD OF INVENTION

The present invention disclosed a novel crystalline form of (R)-terbutaline hydrochloride salt, its preparation and therapeutically applications thereof. The enantiomeric excess (e.e) and chemical purity of as said (R)-terbutaline hydrochloride are 99.9% and 99.7%, respectively. The X-ray powder diffraction diffractogram of as said (R)-terbutaline hydrochloride is characterized by peaks at 2θ of 10.98±0.2°, 12.67±0.2°, 17.53±0.2°, 18.94±0.2°, 19.11±0.2°, 19.4±0.2°, 19.8±0.2°, 21.43±0.2°, 22.12±0.2°, 23.72±0.2°, 25.58±0.2°, 28.91±0.2°, 30.86±0.2°, 31.54±0.2°, 33.48±0.2°. This invention disclosed the therapeutically uses of pharmaceutically acceptable formulation this crystalline form of (R)-terbutaline hydrochloride for the treatment of asthma, COPD, emphysema, lung fibrosis and mucus hypersecretion, lung inflammatory disease and auto-immune diseases.

### BACKGROUND OF INVENTION

Terbutaline is a selective (32 adrenergic receptor agonist which has been widely used for the relief of bronchospasm in asthmatic and COPD patients. Terbutaline is a chiral drug, which consists of two enantiomers: R-isomer and S-isomer. Terbutaline as racemic mixture was first marketed by AstraZeneca in 1988. Currently all the marketed terbutaline products are racemic and in sulfate salt. There were several types of crystalline form of terbutaline have been reported namely: hemi-sulfate hydrate, monohydrate A and B, acetic acid solvate and a higher hydrate. (Acta. Cryst., 1982, B38; Acta. Cryst., 1996, C52, and CN101475497B).

(R)-terbutaline is much more potent than (S)-terbutaline which has no affinity for β adrenoceptor. In addition, (S)-terbutaline was found to be toxic and to cause adverse effects. (Biochim. Biophys. Acta., 2016 Vol. 1858(11); Int. Immunopharmacol., 2019 Vol. 73).

Several methods of manufacturing of optically pure (R)-terbutaline have been reported including: chiral separation using tartaric acid, asymmetric chiral synthesis, chemo-enzymatic, or using chiral selector (β-cyclodextrin or its derivatives). However, most of the previous studies, the finished product, i.e. (R)-terbutaline, was not well characterized in term of chiral structure, enantiomer purity and chemical purity. In addition, all the finished product of (R)-terbutaline or its salts manufactured by method above seemed to be amorphous and no crystalline form has been reported. It is obvious that the crystalline form of (R)-terbutaline is not readily, for a person in art, to resolve as its racemic.

To our knowledge, the crystalline forms of (R)-terbutaline either in free base of in salt has never been disclosed in prior art.

### DETAIL OF THE INVENTION

The present invention provides a novel crystalline form of (R)-terbutaline hydrochloride with both high optical purity and chemical purity. The enantiomeric excess (e.e) and chemical purity of (R)-terbutaline hydrochloride are more than 99.9% and 99.7%, respectively. The crystalline form was needle-like and The X-ray powder diffraction diffractogram of (R)-terbutaline hydrochloride is characterized by peaks at 2θ of 10.98±0.2°, 12.67±0.2°, 17.53±0.2°, 18.94±0.2°, 19.11±0.2°, 19.4±0.2°, 19.8±0.2°, 21.43±0.2°, 22.12±0.2°, 23.72±0.2°, 25.58±0.2°, 28.91±0.2°, 30.86±0.2°, 31.54±0.2°, 33.48±0.2°. The processes for preparing the crystalline form of amorphous (R)-terbutaline hydrochloride comprises:
(a) Amorphous product of (R)-terbutaline hydrochloride salt.
(b) Suspending or forming a slurry of (R)-terbutaline hydrochloride in solvent, then stirring for 2-12 h at 25-70° C.
(c) crystallization the product at temperature from −10 to 10° C. c) recovering the crystalline form product.

In the last step of synthetization of (R)-terbutaline hydrochloride salt involves in hydrochloric acid ethanol as previous reported, the final product was usually in amorphous states. The invention disclosed methods for preparation of the crystalline from the amorphous product of amorphous (R)-terbutaline hydrochloride. The method involved in using mixture of organic solvent of one or two or three of each following solvent with or without adding water: anhydrous ethanol, acetone, hexane, and ethyl acetate, methanol and acrylonitrile. The mixed ratio of for each of the organic solvent is from 5%-95%. The ratio of (R)-terbutaline hydrochloride and total volume of solvents and water if added was preferable between lg:50-100 ml. The slurry time was 4-12 hours at temperature of 25-70 C. The temperature for crystallization was between −10 to 10 C. This invention disclosed that the crystalline form of (R)-terbutaline hydrochloride obtained by above method was needle-like powder or mixed shapes. The impurities were very low and less than 0.1%-0.5%. The above steps and temperatures and ratios were not reported before for resolving (R)-terbutaline hydrochloride crystal. It cannot be anticipated by person in art.

This invention disclosed that the crystalline form of (R)-terbutaline hydrochloride can be processed in micronized form. the crystalline form of R-terbutaline hydrochloride is relatively resistant to agglomeration when micronized, and has a good fluidity. This invention disclosed the crystallization of other (R)-terbutaline salt with mixed solvates at different ratio. In most of the case, crystalline cannot be resolved by forming a hydrochloride salt of (R)-terbutaline unless a proper mixed solvates at a proper concertation of (R)-terbutaline, under suitable temperature for the resolving of crystals. This invention disclosed new uses of the crystalline form of (R)-terbutaline hydrochloride against asthma, COPD, emphysema, lung fibrosis and mucus hypersecretion, acute respiratory distress syndrome. It also can be used for anti-immuno-inflammatory medicine to treatment lung inflammation and chronic bronchiolitis, which may be related to asthma/COPD. Furthermore, It can also be used to treat lung immuno-inflammatory conditions which may be related to other diseases such as HIV infections, tuberculosis, chronic bronchiolitis, idiopathic 100 pneumonia, interstitial lunge diseases (ILD), idiopathic pulmonary fibrosis (IPF), extrinsic allergic alveolitis (EAA), lung cancer or septic shock and systemic infection. auto-inflammatory disease including IBD (inflammatory Bowel Diseases), psoriasis, eczema, urticaria.

This invention disclosed beneficial effects of combining muscarinic receptor antagonist with the crystalline form of (R)-terbutaline hydrochloride. In the combination therapy of crystalline form of (R)-terbutaline hydrochloride with ipratropium or other muscarinic receptor antagonist, using R-enantiomer instead of racemic results in significant better effects and much more reduced adverse effects.

The same beneficial effects were also found when corticosteroids such as budesonide or fluticasone propionate was used instead of muscarinic receptor antagonists.

The muscarinic receptor antagonist according to this invention including tiotropium, glycopyrronium, aclidinium and umeclidiniumt.

The corticosteroids according to this invention include ciclesonide, beclomethasone, mometasone, dethnide, flunisolide, triamcinolone acetonide and other pharmaceutically used corticosteroids or their physiological accept salts and/or solvate thereof according to this invention.

This invention disclosed beneficial effects of combining crystalline form (R)-terbutaline with anti-inflammatory agents such, as montelukast and interferon alpha.

The pharmaceutical preparation of the crystalline form (R)-terbutaline as listed above according to this invention includes syrups or solid dose forms: tablet, hard or soft gelatin capsule or granule for oral use; gel, suppository for rectum or vagina, eye drop, liquid or lyophilized powder for injection; ointment or patches for topic use and aerosol or dry powders for inhalation into lung and nasal.

### EXAMPLES

Method

Burker D8 Advance diffractometer, equipped with Ni-filtered Cu-Kα radiation, was used for the X-ray powder diffraction work. An angular range of 5 to 60° in 2θ was covered.

HPLC analyses were performed on a SPD 20A system (Shimadzu, Japan) that comprised a Shimadzu SPD 20A UV detector, a Shimadzu LC-20AT pump, and a Shimadzu LC solution workstation for the analysis of ee %. The column used for the analysis was a Daicel CHIRALCEL OJ-H column (4.6×250 mm, 5 μm). The wavelength of UV detection was 276 nm. The mobile phase was hexane:ethanol:trifluoroacetic acid:triethylamine mixture (95:5:0.05:0.02). The flow rate was 1.0 mL/min. The analyses were conducted at ambient temperature.

The RP-HPLC system used to investigate the chemical purity and quantification of (R)-terbutaline hydrochloride was a SPD-M20A (Shimadzu, Japan) that comprised a Shimadzu SPD-M20A detector, a Shimadzu CBM-20A controller, two Shimadzu LC-20AT pumps, and a Shimadzu LC labsolution workstation. The chromatographic colunm used for analysis was an Agilent ZORBAX Eclipse XDB-C18 (4.6×150 mm, 5 μm). The mobile phase was composed of 4.23 g sodium hexanesulfonate and 3.15 g ammonium formate in 1000 mL water (adjusted pH to 3.00±0.05 with 10% phosphoric acid)-methanol (77:23, v/v). The temperature during analysis was kept at 40° C. The flow rate was 1.0 mL/min. UV detection was performed at 276 nm.

### Example 1

Preparation of (R)-Terbutaline Hydrochloride in Crystalline Form

1 g of amorphous (R)-terbutaline hydrochloride (purity:>90.0%) was placed in a 200 mL flask. Then 100 ml of solution of hexane and ethanol (v/v: 2:1) was added, the then stirred for 8 h, at temperature 60 C; left to crystallize at 0 C. The crystalline of (R)-terbutaline hydrochloride 0.7 g was then filtered, and dried at 60° C. (yeild: 70%) Purity of the product by RP-HPLC: 99.72%

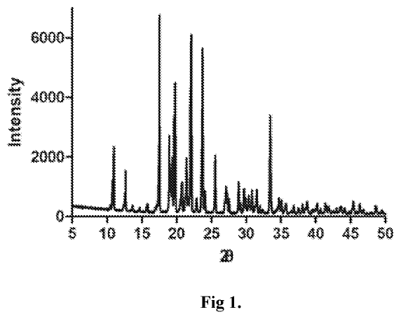

XPRD diffractogram of the product is shown as FIG. 1 and table 1

### Example 2

Preparation of (R)-Terbutaline Hydrochloride in Crystalline Form

1 g of amorphous (R)-terbutaline hydrochloride (purity>90.0%) was placed in a 200 mL flask. Then 100 ml of solution of ethanol and acetone (v/v: 3:1.8) was added, then stirred for 6 h, at temperature 50 C, left to crystallize at 4 C. The crystalline form of (R)-terbutaline hydrochloride 0.62 g was then filtered, and dried at 60° C.(yield: 62%) Purity of the product by RP-HPLC: 99.82%

### Example 3

Preparation of (R)-Terbutaline Hydrochloride in Crystalline Form

1 g of amorphous (R)-terbutaline hydrochloride (purity:99.0%) was placed in a 200 mL flask. Then 100 ml of solution of ethyl acetate and water (v/v: 50:1) was added, then stirred for 8 h, at room temperature, left at 4 C to crystallize. The crystalline of (R)-terbutaline hydrochloride 0.56 g was then filtered, and dried at 60° C. (yeild: 56%) Purity of the product by RP-HPLC: 99.79%

### Example 4

The representative X-ray powder diffraction diffractogram of the finished product above

TABLE 1
(FIG. 1)

| 2θ | d (Å) | intensity (%) |
|---|---|---|
| 10.979 | 8.0523 | 13.4 |
| 12.669 | 6.9813 | 11.9 |
| 17.527 | 5.0559 | 46.6 |
| 18.942 | 4.6811 | 21.4 |
| 19.112 | 4.6399 | 24.9 |
| 19.402 | 4.5713 | 16.6 |
| 19.807 | 4.4788 | 48.9 |
| 21.431 | 4.1428 | 21.4 |
| 22.124 | 4.0146 | 100.0 |
| 23.724 | 3.7473 | 48.9 |
| 25.584 | 3.4790 | 18.5 |
| 28.913 | 3.0855 | 9.9 |
| 30.863 | 2.8948 | 6.3 |
| 31.544 | 2.8339 | 9.6 |
| 33.483 | 2.6741 | 32.0 |

1. A crystalline form of (R)-terbutaline hydrochloride is characterized by a powder XRD pattern with peaks at degrees 2 theta. of 10.98±0.2°, 12.67±0.2°, 17.53 ±0.2°, 18.94±0.2°, 19.11±0.2°, 19.4±0.2°, 19.8±0.2°, 21.43±0.2°, 22.12±0.2°, 23.72±0.2°, 25.58±0.2°, 28.91±0.2°, 30.86±0.2°, 31.54±0.2°, 33.48±0.2°. and a process for preparation of (R)-terbutaline hydrochloride crystalline, and its therapeutically uses for a patient in need.

2. The compound of claim 1, characterized by a powder X-ray diffraction pattern in which the peak positions are substantially in accordance with the peak positions of the pattern shown in FIG.

3. The compound of claim 1, wherein the said (R)-terbutaline hydrochloride crystalline has enantiomer excess value of 95%-99.9%.

4. The process of claim 1, wherein the said process including:
a) preparing amorphous (R)-terbutaline hydrochloride in one or mixed organic solvent with/without adding water;
c) crystalizing the product at temperature from −10 to 25° C.

5. The organic solvent according to claim 4, wherein the said organic solvent comprises one or two of anhydrous ethanol, acetone, hexane, ethyl acetate and acrylonitrile.

6. The mixed organic solvent according to claim 4, wherein the mix ratio for each chosen solvent is from 5% to 95%.

7. The therapeutically uses of claim 1, wherein the said therapeutically used are for the treatment of asthma, COPD, lung inflammation and chronic bronchiolitis, emphysema, lung fibrosis, mucus hypersecretion, acute respiratory distress syndrome and auto-inflammatory disease including IBD, psoriasis, eczema, urticaria.

8. The therapeutically uses of claim 1, wherein said therapeutically uses comprising a therapeutically acceptable carrier and a therapeutically effective amount of a compound of claim 1.

9. The therapeutically uses of claim 1, wherein said therapeutically uses are formulated as syrups or solid dose forms: tablet, hard or soft gelatin capsule or granule for oral use; as gel, suppository for rectum or vagina; as eye drop; as liquid or lyophilized powder for injection; as ointment or patches for topic use and as aerosol or dry powders for inhalation into lung and nasal.

10. The therapeutically uses of claim 1, wherein the therapeutically uses Is in combining with anti-inflammatory agents such, as montelukast and interferon alpha; with ipratropium and tiotropium or with budesonide or fluticasone propionate.
